(19) United States
(12) Patent Application Publication
(10) Pub. No.: US 2014/0058226 A1
(43) Pub. Date: Feb. 27, 2014

(54) Method and Apparatus for In Vivo Optical Measurement of Blood Glucose Concentration

(71) Applicants: Boris M. Chernobrod, Santa Fe, NM (US); Vladimir Schwartz, Lexington, MA (US)

(72) Inventors: Boris M. Chernobrod, Santa Fe, NM (US); Vladimir Schwartz, Lexington, MA (US)

(21) Appl. No.: 13/996,656

(22) PCT Filed: Dec. 22, 2010
(86) PCT No.: PCT/US10/61885

§ 371 Date: Nov. 5, 2013

(51) Int. Cl. A61B 5/145; A61B 5/1455

(52) U.S. Cl. CPC A61B 5/14532; A61B 5/1455
USPC 600/316

(57) ABSTRACT

A method of non-invasive measurement of the glucose concentration directly in the blood flow utilizes a combination of the differential scattering spectroscopy and confocal scanning laser Doppler microscopy.

### RELATED APPLICATIONS

This Application is a National Stage application of International Application PCT/US2010/061885, filed on Dec. 22, 2010, which is incorporated herein by reference in its entirety.

### FIELD OF THE INVENTION

The present invention is related to the field of non-invasive optical detection and measuring of glucose concentration in blood vessels.

### BACKGROUND OF THE INVENTION

It is well established that a measurement of glucose concentration directly in blood vessels is the most reliable method for the monitoring of diabetic metabolism. Many methods and devices have been developed till now for the determination of glucose in vitro or in vivo by optical means. Progress towards the development of blood glucose monitoring methods is disclosed in. Light scattering from the red blood cells (RBCs) is one of the noninvasive in-vivo blood glucose monitoring methods. This method exploits the fact that a change in glucose concentration leads to the change in the scattering coefficient of red blood cells. A major obstacle for high accuracy measurement is a parasitic scattering of light radiation in the skin tissues. Additionally, light scattering in tissues is influenced by the glucose concentration as well. Yet, the glucose concentration in tissues is not a direct manifestation of, glucose metabolism. Rather, it is a function of the local blood flow velocity, tissue temperature, oxygenation rate, etc. Additionally, other tissue constituents could influence light scattering as well. For example the glucose signal can be masked by the water absorption. Significant changes in skin tissue water concentration (±20%), can lead to unacceptably low accuracy and reproducibility of the blood glucose concentration measurements. Note, that the variation of water concentration in blood is relatively small (±1.8) The U.S. Pat. No. 5,137,023, and US published applications 20060063983, 20080027297 disclose a technique that eliminates various influences of the skin tissues and water absorption. This invention exploits the method of differential spectroscopy by using the laser light radiation with two spectrally close wavelengths. The difference or ratio of the signals at these two wavelengths is independent of the water absorption and/or other biological and chemical substances. The elimination of influence by the substances is possible since the selected wavelengths correspond to sharp features in the glucose spectra. The separation of the signal from blood and tissue is based on the electronic filtering of the heart bit modulation. However, the experimental realization of this method has demonstrated unacceptably low reproducibility of the measurements due to the influence of glucose in present in skin tissues.

### SUMMARY OF THE INVENTION

The invention describes a method of non-invasive measurement of the glucose concentration directly in the blood flow by utilizing a combination of the differential scattering spectroscopy and confocal scanning laser Doppler microscopy. The main sources of the irreproducibility of glucose optical measurements are the influences of skin tissues glucose and water absorption. The differential spectroscopy method exploits the measurement of backscattering from the red blood cells (RBC) in micro-vessels by using two coaxial laser beams at two wave lengths inside of the water absorption window (for example 2190-2500 nm or 1000-1560 nm). Inside of the absorption windows the RBC scattering has relatively sharp resonance features corresponding to the influence of the vibration resonances of glucose on mismatch of refractive index (for example, combination of the vibration resonance at 2300-2500 nm, and first-overtone resonances at 1000-1560 nm). The selected laser wavelengths are located near the local maximum and minimum of the scattering coefficient correspondently. To avoid of an influence of the water absorption on the extinction coefficient, the wavelengths of the lasers are located at the symmetrical positions relative to the local minimum of water absorption. Thus, the difference or ratio of two backscattering signals is independent on the water absorption. In order to distinguish between the light scattering in tissues and blood, we apply a method of confocal scanning Doppler microscopy (CSDM). In this method, two coaxial laser beams are focused inside of the blood vessels. The backscattering beams are separated by the dispersion element such as beam splitter, dichroic mirror, grating, or Fabry-Perot resonator or other optical elements and each beam mixes with a reference beam in the interferometer. The backscattering signal includes a frequency shifted optical signal due to the scattering from moving RBC and a frequency non-shifted optical signal due to scattering from skin static structures. The interference signal oscillating at the Doppler frequency produces an alternating current (AC) which is detected by the photodetector. The backscattering signals including the signal at the Doppler shifted frequency, propagate through skin tissue layers such as dermis, epidermis and stratum corneum (see FIG. 2). These tissue layers also contain glucose and other bio-chemical constituents which affect the extinction coefficient in a way similar to the light scattering in blood. Thus, the Doppler shifted signal acquires additional features leading to the measurement error and irreproducibility. The specificity of these features is that they are produced by the static (or unmovable) structures. In order to identify this influence, we can measure a non-oscillating part of the signal responsible for direct current (DC). By learning the dependence of DC signal on the depth of laser beam penetration, we can measure an influence of skin tissues on AC signal. The dependence of the differential DC signal on Doppler frequency is negligibly small. Therefore, we can account for only the influence of skin tissues on the AC signal. To eliminate a low frequency noise, the frequency of the reference signal can be shifted by the frequency shifter. In this case, the scattering back signal from the static structures with non-shifted frequency also induces AC current of the photodetector at a different frequency when compared with the Doppler shifted signal.

Yet another advantage of the differential confocal spectroscopy is the suppression of the signal fluctuations related to RBC motion, beam scanning over the inhomogeneous tissues and skin movements. These sources of fluctuations affect the signals at both wavelengths in the same manner due to two coaxial focusing of laser beams which interact synchronously with the same RBC or tissue micro-volume. In other words, the fluctuations of the signals are strongly correlated in time. Thus, the relative difference or ratio of two signals is independent on these fluctuations.

### BRIEF DESCRIPTION OF THE DRAWINGS

FIG. 1 is the schematic illustration of the measurement methodology.

FIG. 2 is the schematic illustration of the light skin interaction.

FIG. 3 is the schematic illustration of the optimal wavelengths position relative the urea spectrum peak

FIG. 4 is the illustration of the conformal surface immersion

### DETAILED DESCRIPTION OF THE INVENTION

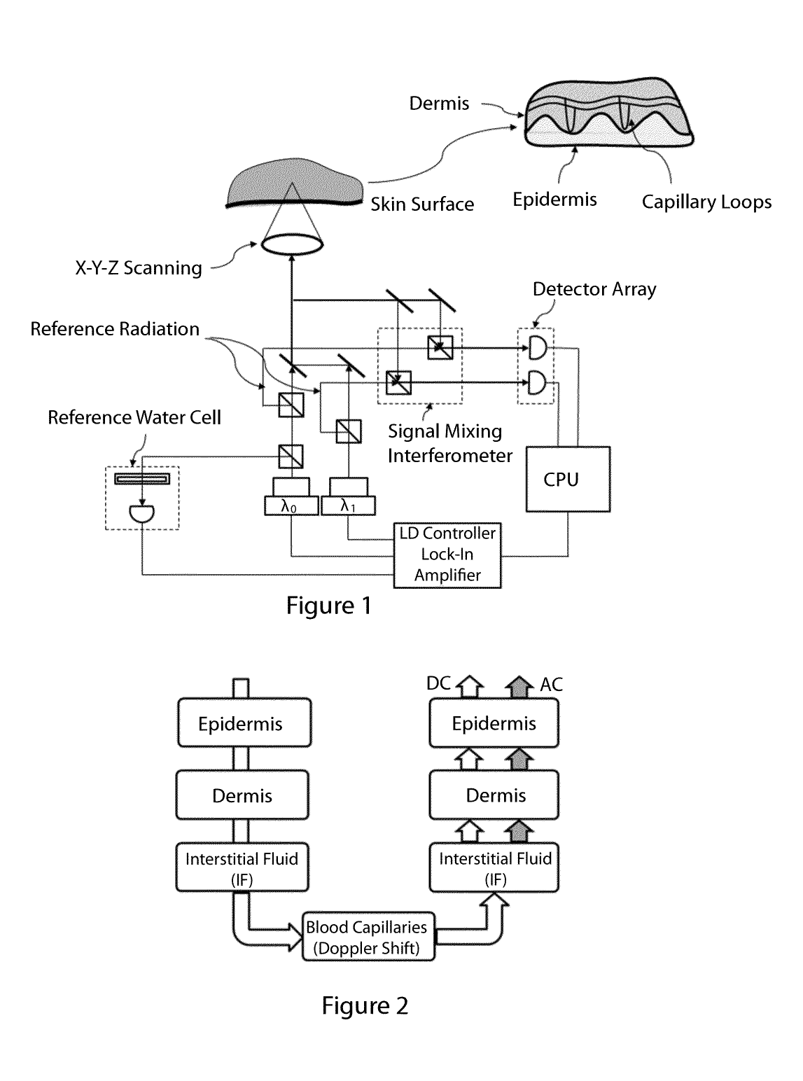

The block-diagram of the measurement methodology is presented in FIG. 1. A small portion of, light from two lasers beams with the wavelengths λ₀ and λ₁ (emanating from a tunable laser source or sources) is sent towards the water reference cell. The water reference cell temperature is stabilized and is equal to the skin tissue temperature. The wavelength of both laser beams is tuned to the symmetric position relative to the center of the selected water absorption window. Therefore, the water absorption is the same for each wavelength and the difference or ratio of the backscattering signals is independent of the water absorption. In the next step, the main portion of the laser power is split into the probing and reference beams. The reference beam(s) is sent to the interferometer. The probing beams are arranged coaxially. To average out and reduce a parasitic influence of the speckle structures on the backscattering signal, the excitation beams are transformed into partially coherent beams by utilizing a rotating phase diffuser, deformable mirror and/or other anti-speckle devices. Partially coherent beams are focused inside of the skin in such a way, where the beam waist is located inside of the region of blood vessels. To average out the skin inhomogeneous structures and to extend the limits of maximum permissible power exposure or MPE (according to the American National Standard for safe use of lasers ANSI+Z136.1), the laser beam(s) is x-y-z scanned. Servo positioning and scanning of the objective lens relative to the skin surface enables selective detection of the test volume within dermis. AC signal will be utilized for the objective lens positioning (servo feedback loop). Traversing objective will find the maximum AC signal within the dermis layer. The backscattering signals are angularly separated by the beam slitter, wavelength dispersion element or elements (such as dichroic mirror, grating or Fabry-Perot resonator, etc.) and each beam is sent to its own interferometer or one common interferometer. The interference signals are detected and processed. Being an instrument for the separation of the Doppler shifted signal, the interferometer provides two additional advantages. The first advantage is the homodyne enhancement of the signal. The second advantage is an additional spatial selectivity of the signal. Only the signal waves propagating back along optical axis contribute efficiently to the interference signal. These waves emerge from the beam focus waist. Thus, the interference suppresses a parasitic background signal. This background signal includes multiply scattered photons and photons emerging outside of the focus volume. Both types of photons are very undesirable because they lead to signal misinterpretation. The blood glucose concentration is measured by detecting the differences in the scattering due to changes of refractive index mismatch between RBCs and surrounding blood volume (plasma) or the difference in absorption coefficients. Refractive index of RBCs is larger than refractive index of blood, n₁>n₂ (n₁˜1.4, n₂˜1.35). The refractive index mismatch causes photon scattering. Due to the movement of RBCs, the scattering is Doppler frequency shifted (AC signal, Δfₘₐₓ≦20 KHz). Low frequency signals due to heartbeat, vasomotions, muscle movements, etc. will be filtered out. An influence of the water on the signal reproducibility is low since water concentration in the blood volume varies by ±1.8 percent (water concentration in skin tissues varies by ±20 percent). Glucose scattering qualities are universal for diabetics and non-diabetics.

Another embodiment comprises one interferometer and no dispersion element(s) for the angular signal separation. The radiation is provided by the consecutive pulses of two lasers. The repetitive rate of each laser is much faster than any character fluctuation time of signal.

Another embodiment comprises one interferometer, no dispersion elements for the angular signal separation, and one laser. The radiation is provided by consecutive pulses at different wavelength. The pulse repetitive rate of laser is much faster than any character fluctuation time of signal.

Another embodiment comprises one interferometer, beam splitter and no dispersion element(s) for the angular signal separation. The reference beams of each wavelength interfere only with a part of the signal at its own wavelength.

### Technical References

### Resonance Absorption and Anomalous Refractive Index

The term “complex refractive index” is:

$${n}^{2} = {n}_{0}^{2} + {\sum }_{j}^{ } \frac{{α}_{j} {Γ}_{j}^{2}}{{ω}^{2} - {ω}_{j}^{2} + i {Γ}_{j} ω}$$

Where, j is the resonance number.

The absorption coefficient is:

$$α ( ω ) = Im \{ \frac{ω}{c} [ {n}^{2} ( ω ) - {n}_{0}^{2} ] \}$$

The addition part of the refractive index in case of weak dispersion is:

$$n = R e \frac{1}{2} [ {n}^{2} ( ω ) - {n}_{0}^{2} ]$$

Importantly, spectral resonance features appear in the absorption coefficient and refractive index simultaneously.

Major Blood Analytes and their Influence on the Refractive Index

In the case of relative thin/shallow turbid media, the backscattering signal Pₛ is proportional to the reduced scattering coefficient μ′ₛ(1−g), where g is the anisotropy factor Pₛ˜μ′ₛ. The quantity of interest is δμ′ₛ/μ′ₛ. Where the symbol δ means the differential δμ′ₛ=μ′ₛ(λ₁)−μ′ₛ(λ₀).

The wavelength dispersion of the reduced scattering coefficient can be divided into two parts. The first part δμ′ₛ₀ is related to the geometrical form and size of RBC and is a universal property of Mie-like scattering. The second part δμ′_{sg} appears due to the dispersion of the refractive index and depends on the contribution of analytes. The spectral resonances of glucose can contribute to the second part. This means that the second part depends only on the glucose concentration.

### Ballistic Approximation

In the case of relatively thin/shallow turbid media, the main contribution to the backscattering signal produced by the photons which have experienced only a single act of scattering. In this assumption, we can consider the propagation of the plane waves in the media with a certain extinction coefficient μₜ. The scattering intensity for Doppler (AC) and static (DC) signals are given by:

P_{AC,DC}=ΔΩμ′_{RBC,ST}L_{c}exp[−=∫₀^{z}dzμₜ(z)] (1)

where ΔΩ is the spherical angle, I_{L} is the initial laser intensity, L_{c} the beam waist length in the confocal geometry, μₜ(z)=μ′_{RBC}(z)+μ′_{ST}+μₐ is the total light scattering coefficient including the coefficients of scattering from red blood cells (RBC) and from static structures (ST), and the absorption coefficient μₐ. The quantity of interest is

$$\frac{{δμ}_{RBC}^{′}}{{μ}_{RBC}^{′}}$$

where, μ′_{REC} is the isotropic part of light scattering coefficient from red blood cells δ μ′_{REC}=λ′_{RBC}(λ₁)−μ′_{RBC}(λ₀).

According to Equation (1) we have:

$$\frac{δ {P}_{AC}}{{P}_{AC}} = \frac{{δμ}_{RBC}^{′}}{{μ}_{RBC}^{′}} - 2 {\int }_{0}^{z} d z {δμ}_{t} ( z ) , \\ where \\ δ {P}_{AC} = {P}_{AC} ( {λ}_{1} ) - {P}_{AC} ( {λ}_{0} ) \qquad ( 2 )$$

Thus, it is possible to derive from Eq. (1) that:

$$δln \frac{{P}_{DC} ( z )}{{P}_{DC}} = - 2 {\int }_{0}^{z} d z δ {μ}_{t} ( z ) . \qquad ( 3 )$$

Finally we get:

$$\frac{{δμ}_{RBC}}{{μ}_{RBC}} = \frac{δ {P}_{AC}}{{P}_{AC}} + δln \frac{{P}_{DC} ( z )}{{P}_{DC}} \qquad ( 4 )$$

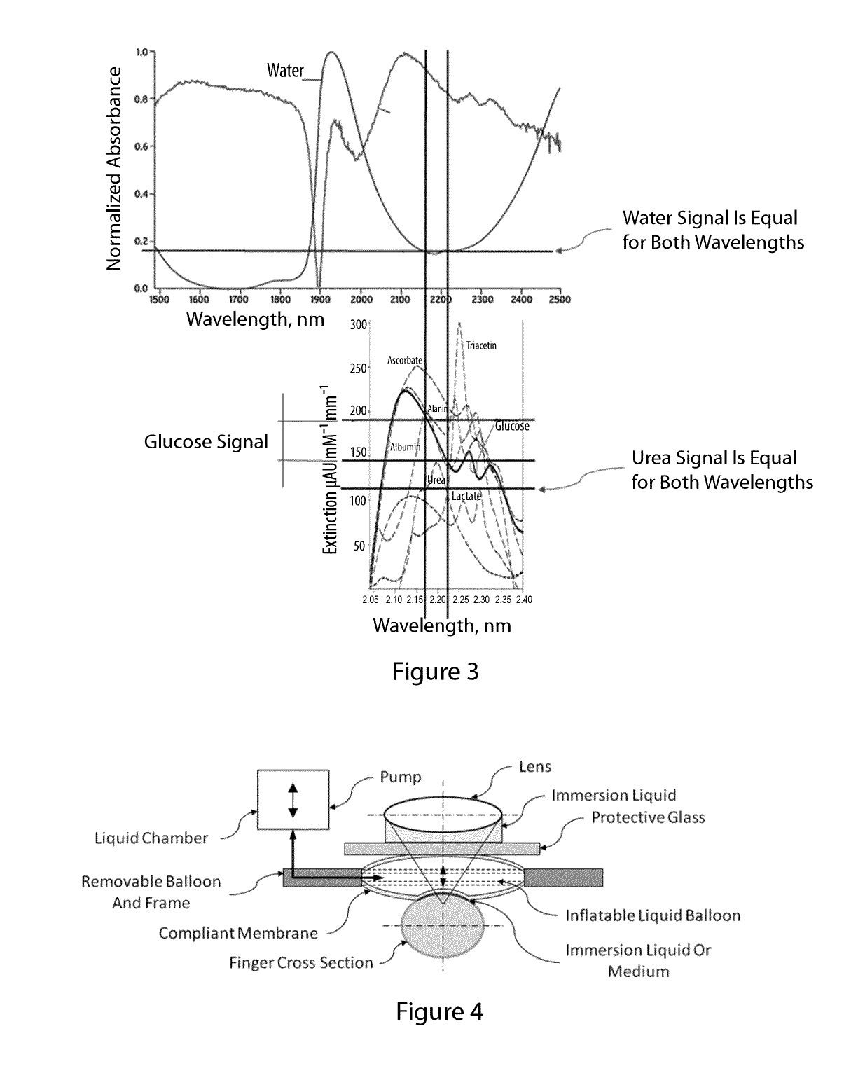

In order to eliminate an influence of the epidermis and stratum corneum, we employ the principles of confocal microscopy. The idea behind the utilization of confocal principles in the direct measurements of the blood glucose concentration is to eliminate an influence of glucose and static structures present in the epidermal layer (which contains no blood vessels), see FIG. 2. The epidermal structures and its glucose surround the blood micro-vessels from within which the scattering signal influenced by the blood glucose is originated. Light scattered by the moving erythrocytes (red blood cells or RBCs) propagates through the skin tissues and adds the spectral features similar to those present in the blood itself. The static epidermal tissue structures and its glucose act as a parasitic spectral filter for the light scattered by the RBCs. The significant difference, however, is that the signal from static structures (vs. signal from RBCs) is non-reproducible and can't be utilized to assess the glucose metabolic state or its concentration. The fact that the propagating light “remembers its “history” is demonstrated by formula (I). This formula has an exponential factor exp [−2∫₀^{z}μₜdz], which is an integral over the entire optical path. To eliminate this effect we utilize two signals. The first one is the Doppler shifted signal from RBCs, which produces bits on the photodetector (AC signal). The second signal is produced by the static scattering structures and has no Doppler shift (DC signal), or has a different AC frequency in the case when the reference beam frequency is shifted by the frequency shifter. Both signals have originated from within the same measurement volume defined by the confocal beam waist. Propagating along the same optical path, these two signals acquire the same parasitic spectral features. In other words, the exponential factor related to the optical path is the same for both signals. By using an analytical combination of the measured intensity, we eliminate the parasitic propagation effects. We suggest the formula for the signal processing (see Equation 4), which doesn't depend on the exponential factor and is free of the parasitic influence of the static scattering structures and water absorption. According to the calculations based on Mie theory and measurements using optical coherence tomography (OCT), the major contributors in the reduced scattering at the infrared region (2200-2500 nm, 1000-1560 nm) are glucose, urea, NaCl and KCl. If the contribution of glucose is equal to unity, then the contribution of urea is 7.3%. The concentration changes of NaCl and KCl are minor and their spectral contribution is not specific in the spectral region of interest An influence of urea on the refractive index mismatch can be reduced to 0.5% or less (from the original 7.3%) by selecting an appropriate spectral region. The selected of the spectral region is demonstrated in FIG. 3, where the absorptivity spectra of glucose and urea are presented using data from the paper Larin, K. V. et al. “Specificity of noninvasive blood glucose sensing using optical coherent tomography: pilot study”, Phys. Med. Biol., vol. 48 (2003) 1371-1390. The wavelengths of the lasers are chosen to be symmetrical relative the spectral peak of urea. Thus, the difference or ratio of the signals is practically independent on the urea concentration.

### Calibration Procedure

As a part of assembly procedure, glucose meters will undergo the electronic and optical calibration. Meters will utilize the glucose calibration matrix. The data matrix will be established by matching the glucose signal of calibration test stand with the glucose concentration. The calibration procedure can use the glucose measurement in vivo at different level of blood glucose concentration. Also calibration is possible by using measurement in vitro where whole blood, plasma or serum samples within the hypo to hyper glycemic range. Glucose concentration will be measured by high precision analyzer such as YSI 2300 STUT Plus, YSI Life Sciences (±2% accuracy). The calibration test stand will be based on the glucose meter design, but will have an additional capability to test and/or measure the specified parameters of critical performance components.

### Additional Features

### Speckle Reduction

Usually laser light propagating through the tissue acquires a speckle structure due to the statistically independent scattering from various tissue structures. To avoid the undesirable signal fluctuations due to the time dependent speckle structure, we utilize of a partially coherent beam (PCB) with the time dependent structure of coherent spots. PCB is organized by the transmission (reflection) of coherent laser beam via the light spatial phase modulator (SLM). SLM forms a PCB by inserting in the laser phase front a time dependent phase structure in the form of statistically independent phase spots. PCB produces time dependence in the speckle structure of scattering signal emerging from the tissue. Time averaging of this time dependence by the photo-detector leads to a suppression of the signal speckle structure. PCB allows deeper skin penetration.

As an SLM we can utilize electro optical phase modulator, rotating or shifting phase diffuser, deformable mirror, etc.:
a) Another method of the time averaging of the speckle structure could be a spatial scanning of the laser beam over the tissue surface, and scanning of the laser beam focus in a direction perpendicular to tissue surface.
b) Another method of the speckle structure suppression could be the utilization of adaptive optics devices, which introduce the correction of the laser phase front in order to avoid the origination of speckle structure.

### Time Averaging of the Signal Fluctuations Due to the Inhomogeneous Tissue Structures

Spatial inhomogeneous tissue structures such as blood micro-vessels, fibers and individual blood cells may lead to certain signal fluctuations in time during the scanning of laser beam in the tissue. These fluctuations are suppressed by the time averaging during the signal processing. The averaging procedure includes a special algorithm of the spatial scanning, signal filtering and modulation of the reference beam in the interferometer.

### The Accuracy Improvement of the Wavelength Tuning Via the Reference Cells

To obtain a high accuracy of the laser wavelength position, we introduce a set of thermally controlled reference microcells (within the range of +/−0.1° C.). These optically transparent microcells can include such substances as water, water solution of glucose, water solution of urea and possibly other blood components. The temperature control of these reference cells will be in a closed-loop arrangement with the device to measure the temperature of skin tissues such as epidermis and dermis.

### Laser Wavelength Calibration

System may contain a gas absorption cell (for example filled with carbon monoxide) for the purpose of the laser wavelength verification, control and calibration.

### Skin Fresnel Reflections

System envisions a set of optical components and/or arrangements to substantially reduce the effects of laser Fresnel reflections arising from the skin surface. Such reflections reduce the measurement accuracy of the instrument. In order to further reduce the Fresnel reflections and other optical losses due to an air/skin interface, we can utilize a method of optical surface immersion (FIG. 4). The proposed inflatable immersion balloon with pre-selected refractive index matched materials (liquid and membranes) accomplishes this goal. Skin surface requires a layer of immersion liquid as well (fills in the gaps, etc.). Compliant membrane helps to prevent various optical changes in the skin tissues due to a mechanical impact (causes the expulsion of tissue water and blood from micro-vessels—both can be a source of glucose signal irreproducibility).

### Instrument Packaging

Environment of the instrument is hermetically sealed to avoid the effects of the air humidity and dust. Humidity will introduce a parasitic absorption by the laser beams and ultimately reduce the measurement accuracy. Dust will introduce various parasitic reflections and thus the deterioration of optical surfaces quality, which leads to various optical aberrations and the reduction of the instrument measurement accuracy.

### The Reduction of the Tissue Background Signal

System contains an arrangement (an additional set of photo-detectors, for example) to substantially reduce/suppress the effects of background signal on the precision of the probing confocal signal.

### Thermal Stabilization of Lasers, Photo-Detectors and Opto-Mechanical Module

System may contain the necessary arrangements and/or control loops to enable the thermal stability of lasers and photo-detectors. Control of the thermal stability of opto-mechanical module is also envisioned to maintain the precise positioning of various optical components (such as interferometers).

### Optical Focusing Arrangement

System envisions the necessary precise position control arrangement of the optical focus (probing beam or beams) relative to the skin surface. Such position control may be enabled via the autofocus, triangulation and/or other optical and opto-mechanical methods.

What is claimed is:

1. An optical method of non-invasive measuring of a concentration glucose in blood comprising:
providing a first laser beam at a first wavelength and a coaxially aligned second laser beam at a second wavelength, the first and the second wavelengths differing by a wavelength interval close to a sharp spectral features of glucose.
directing a portion of both laser beams to a water reference cell and controlling equal absorption in the water reference cell by tuning both wavelengths relative to the center of a water absorption window of the water reference cell;
directing the first beam into the measurement volume along a confocal optical path, scanning the first beam in the measurement volume, and obtaining a first scattered signal at the first wavelength being a Doppler shifted signal scattered from dynamic objects in a blood flow, and obtaining a second scattering non-shifted signal at the first wavelength being a signal from non-blood carrying static structures; directing a second beam into the measurement volume along the confocal optical path, scanning the second beam in the measurement volume, and obtaining a first scattered signal at the second wavelength being a Doppler shifted signal scattered from dynamic objects in a blood flow, and obtaining a second scattering non-shifted signal at the second wavelength being a signal from non-blood carrying static structures;
directing both Doppler shifted and non-shifted signals at the first wavelength to an interferometer and mixing both signals with a first reference beam at the first wavelength, and detecting a first interference signal as an alternate current and direct current;
directing both Doppler shifted and non-shifted signals at the second wavelength to the interferometer and mixing both signals with a second reference beam at the second wavelength, and detecting a second interference signal as an alternate current and direct current;
utilizing signal processing of the first and the second interference signals to obtain a differential signal indicative of a concentration of glucose in blood.

2. The method of claim 1, wherein scanning the first and/or the second beam in the measurement volume comprising X-Y-Z scanning.

3. An optical method of non-invasive measuring of a concentration glucose in blood comprising:
providing a first laser beam at a first wavelength and a coaxially aligned second laser beam at a second wavelength, the first and the second wavelengths differing by a wavelength interval close to a sharp spectral features of glucose.
directing the first beam into the measurement volume along a confocal optical path, scanning the first beam in the measurement volume, and obtaining a first scattered signal at the first wavelength being a Doppler shifted signal scattered from dynamic objects in a blood flow, and obtaining a second scattering non-shifted signal at the first wavelength being a signal from non-blood carrying static structures; directing a second beam into the measurement volume along the confocal optical path, scanning the second beam in the measurement volume, and obtaining a first scattered signal at the second wavelength being a Doppler shifted signal scattered from dynamic objects in a blood flow, and obtaining a second scattering non-shifted signal at the second wavelength being a signal from non-blood carrying static structures;
directing both Doppler shifted and non-shifted signals at the first wavelength to a first interferometer and mixing both signals with a first reference beam at the first wavelength, and detecting a first interference signal as an alternate current and direct current;
directing both Doppler shifted and non-shifted signals at the second wavelength to a second interferometer and mixing both signals with a second reference beam at the second wavelength, and detecting a second interference signal as an alternate current and direct current;
utilizing signal processing of the first and the second interference signals to obtain a differential signal indicative of a concentration of glucose in blood.

4. An optical method of non-invasive measuring of a concentration glucose in blood comprising:
providing a tunable laser source to generate a first laser beam at a first wavelength and a coaxially aligned second laser beam at a second wavelength, the first and the second wavelengths differing by a wavelength interval close to a sharp spectral features of glucose.
directing the first beam into the measurement volume along a confocal optical path, scanning the first beam in the measurement volume, and obtaining a first scattered signal at the first wavelength being a Doppler shifted signal scattered from dynamic objects in a blood flow, and obtaining a second scattering non-shifted signal at the first wavelength being a signal from non-blood carrying static structures; directing a second beam into the measurement volume along the confocal optical path, scanning the second beam in the measurement volume, and obtaining a first scattered signal at the second wavelength being a Doppler shifted signal scattered from dynamic objects in a blood flow, and obtaining a second scattering non-shifted signal at the second wavelength being a signal from non-blood carrying static structures;
directing both Doppler shifted and non-shifted signals at the first wavelength to an interferometer and mixing both signals with a first reference beam at the first wavelength, and detecting a first interference signal as an alternate current and direct current;
directing both Doppler shifted and non-shifted signals at the second wavelength to the interferometer and mixing both signals with a second reference beam at the second wavelength, and detecting a second interference signal as an alternate current and direct current;
utilizing, signal processing of the first and the second interference signals to obtain a differential signal indicative of a concentration of glucose in blood.

5. The method of claim 1, further comprising providing an inflatable immersion balloon of a pre-selected refractive index along the confocal optical path between the confocal arrangement and the measurement volume.

6. An optical method of non-invasive measuring of a concentration glucose in blood comprising:
providing a first laser beam at a first wavelength and a coaxially aligned second laser beam at a second wavelength, the first and the second wavelengths differing by a wavelength interval close to a sharp spectral features of glucose.
directing the first beam into the measurement volume along a confocal optical path, scanning the first beam in the measurement volume, and obtaining a first scattered signal at the first wavelength being a Doppler shifted signal scattered from dynamic objects in a blood flow, and obtaining a second scattering non-shifted signal at the first wavelength being a signal from non-blood carrying static structures;
directing a second beam into the measurement volume along the confocal optical path, scanning the second beam in the measurement volume, and obtaining a first scattered signal at the second wavelength being a Doppler shifted signal scattered from dynamic objects in a blood flow, and obtaining a second scattering non-shifted signal at the second wavelength being a signal from non-blood carrying static structures;
directing, a first portion of the Doppler shifted and non-shifted signals at the first wavelength and a first portion of the Doppler shifted and non-shifted signals at the second wavelength to a first interferometer and mixing both signals with a first reference beam at the first wavelength, and detecting a first interference signal as an alternate current and direct current;
directing a second portion of the Doppler shifted and non-shifted signals at the second wavelength and a second portion of the Doppler shifted and non-shifted signals at the first wavelength to a second interferometer and mixing both signals with a second reference beam at the second wavelength, and detecting a second interference signal as an alternate current and direct current;
utilizing signal processing of the first and the second interference signals to obtain a differential signal indicative of a concentration of glucose in blood.

7. The method of claim 1, wherein detecting the second interference signal as the alternating current of a different frequency when both reference beam frequencies are shifted by a frequency shifter or shifters.
